(19) United States
(12) Patent Application Publication
(10) Pub. No.: US 2023/0241154 A1
(43) Pub. Date: Aug. 3, 2023

(54) COLLAGEN HYDROLYSATE AS AN ACTIVE SUBSTANCE FOR DELAYING AGEING

(71) Applicant: GELITA AG, Eberbach (DE)

(72) Inventors: Stephan HAUSMANNS, Heidelberg (DE); Hans-Ulrich FRECH, Weinheim (DE); Steffen OESSER, Glücksburg (DE)

(21) Appl. No.: 18/131,524

(22) Filed: Apr. 6, 2023

Related U.S. Application Data
(63) Continuation of application No. PCT/EP2021/069480, filed on Jul. 13, 2021.

(30) Foreign Application Priority Data
Oct. 9, 2020 (DE) 102020126594.8

(51) Int. Cl. A61K 38/01; A61P 43/00; A61Q 19/08

(52) U.S. Cl. CPC A61K 38/014; A61P 43/00; A61Q 19/08

(57) ABSTRACT

The present invention relates to collagen hydrolysate for use as an active substance in order to delay the ageing of cells in the bodies of humans or animals.

### FIELD OF THE INVENTION

The present invention relates to collagen hydrolysate for use as an active substance in order to delay the ageing of cells in the bodies of humans or animals.

### BACKGROUND OF THE INVENTION

On the one hand, biological ageing of the body in humans or animals is linked to external environmental influences, which accumulate over a lifetime. However, independently of such environmental influences, at the level of individual cells of the body there is also a general link between ageing and a gradual shortening of telomeres during a lifetime. Telomeres are repetitive sections of DNA at the ends of chromosomes, which initially become shorter each time a cell divides. This shortening may be partly compensated for by the DNA-synthesising enzyme telomerase, the activity of telomerase being very different in different cell types. Once the length of the telomeres falls below a particular value, there are no further cell divisions.

For the ageing process and hence lifespan of an organism, it is not so much the original length of the telomeres that is relevant as the telomere shortening rate, which may be defined for example as the average shortening of telomeres in base pairs per year of life (see K Whittemore et al. in PNAS 116 (2019) 15122-15127).

For some time, active substances that counter the shortening of telomeres have been sought, in order in this way to delay the ageing of cells in the bodies of humans or animals and hence tissues, organs and the body as a whole. Such considerations relate in particular (but not exclusively) to ageing of the skin, since this is particularly outwardly noticeable.

As corresponding active substances for delaying ageing, various plant extracts have already been proposed, for example plants of the genera Astralagus and Cimicifuga in WO 2005/044179 A2 or the genera Dendropanax and Kappaphycus in WO 2018/139835 A1.

### BRIEF SUMMARY OF THE INVENTION

The object of the invention is to propose an effective active substance for delaying the ageing of cells in the bodies of humans or animals.

### DETAILED DESCRIPTION OF THE INVENTION

According to the present invention, to achieve this object the use of collagen hydrolysate is proposed as the corresponding active substance. Cell tests have been able to show that collagen hydrolysate significantly counters the shortening of telomeres and activates telomerase, as is explained in detail below.

In particular, the invention relates to the use of collagen hydrolysate in order to delay the ageing of the skin. This is typically expressed in the form of the formation of wrinkles, loss of elasticity, formation of so-called liver spots, etc. In this case, the cells of the body that are affected by the action of collagen hydrolysate are fibroblasts.

Other types of body cells of which ageing can be delayed within the scope of the invention are other connective tissue cells such as osteoblasts and chondrocytes, and muscle cells (myocytes), nervous system cells (neurons) and cells of the immune system.

In a preferred embodiment of the invention, the use of collagen hydrolysate is a non-therapeutic use, that is to say in particular a cosmetic use. This takes account of the fact that biological ageing of the body, as a natural process, is not a pathological condition that is in need of therapy in the medical sense. Nevertheless, a delay in ageing can contribute to an improvement in the quality of life.

According to a further aspect of the invention, the use of collagen hydrolysate is a therapeutic use to prevent and/or treat age-related diseases. Diseases of this kind that are linked to a shortening of the telomeres are in particular atherosclerosis, chronic liver disease, inflammatory bowel disease and certain diseases of the adrenal gland and spleen. Further diseases that occur cumulatively with increasing age are osteoporosis, arthrosis, sarcopaenia and dementia such as Alzheimer's disease.

In the use according to the invention, collagen hydrolysate preferably counters the shortening of telomeres in the cells of the body, and in particular reduces the telomere shortening rate. As mentioned above, at least when making a comparison between different species, the telomere shortening rate is correlated with lifespan. The cell tests that were carried out in human fibroblasts were able to show explicitly a reduction in this rate by collagen hydrolysate.

Within the scope of the use according to the invention, the collagen hydrolysate can activate the enzyme telomerase in the cells of the body. It was also possible to show this effect in the cell tests with human fibroblasts. However, activation of telomerase is not necessarily the only mechanism for action to counter shortening of the telomeres.

It is true that positive physiological effects from collagen hydrolysate have already long been known, in particular in conjunction with osteoporosis or joint ailments. However, there were no indications at all of an interaction between collagen hydrolysate and telomeres, so the activity observed within the scope of the present invention is entirely surprising.

Depending on whether use of collagen hydrolysate is non-therapeutic or therapeutic, within the scope of the present invention the collagen hydrolysate may be administered as a nutritional supplement or as a medicament. In any case, collagen hydrolysate is a product that does not present any health risks whatever and has no known harmful side effects.

Preferably, the collagen hydrolysate is administered orally. It is known that the peptides of collagen hydrolysate are resorbed in the intestine, at least to a certain extent, even at relatively high molecular weights of up to 10 000 Da. In principle, therefore, orally administered collagen hydrolysate may reach all the cells of the body as the site of action.

The actual form of administration of collagen hydrolysate may be as a powder, a solution, a gel, a tablet or a capsule.

The daily dose of the collagen hydrolysate administered, in particular in the case of oral administration, is favourably from approximately 0.5 to approximately 20 g, preferably from approximately 2 to approximately 15 g, more preferably from approximately 3 to approximately 10 g, in particular a daily dose of from approximately 4 to approximately 8 g.

As the active substance according to the invention, the collagen hydrolysate typically has an average molecular weight of from 500 to 15 000 Da, preferably from 1 000 to 8 000 Da, more preferably from 1 500 to 5 000 Da, most preferably from 1 800 to 2 200 Da. In each case, the figures quoted refer to the weight-average molecular weight, which may be determined in particular by gel permeation chromatography.

Preferably, the collagen hydrolysate is produced by enzymatic hydrolysis of a starting material containing collagen. For this hydrolysis, in particular endopeptidases or exopeptidases of microbial or plant origin are used. Suitable selection of the peptidases and the hydrolysis conditions enables collagen hydrolysates in the respectively desired range of molecular weights to be produced.

The collagen-containing starting material is typically selected from the skin or bones of vertebrates, preferably mammals or birds, and in particular the skin of cattle or pigs (bovine split or pork rind). As an alternative, the starting material containing collagen may be selected from the skin, bones and/or scales of fish, in particular cold-water or warm-water fish.

However, as a starting material collagen hydrolysate may also be obtained from invertebrates and used within the scope of the present invention. For example, collagen hydrolysates may be produced from molluscs or jellyfish.

Collagen hydrolysate can either be produced in a one-step method from these starting materials, or by way of the intermediate stage of gelatine, in which case both type A gelatine and type B gelatine can be used.

Preferably, the collagen hydrolysate is produced by the successive action of at least two endoproteases of different specificities, in particular at least two different metalloproteases and/or serine proteases, that is to say of proteases that break the amino acid sequence of the collagen molecules respectively before and after certain amino acids. Favourably, the metalloproteases and/or serine proteases are enzymes from the microorganisms Bacillus subtilis, Bacillus licheniformis, Bacillus amyloliquefaciens, Aspergillus oryzae and Aspergillus melleus.

As a result of selecting suitable endoproteases, it is not only possible to obtain a particular molecular weight distribution of the collagen hydrolysate, but also to affect the type of amino acids at the ends of the peptides in the hydrolysate. In this regard, it is preferable for example if at least 50% of the N-terminal amino acids of the collagen hydrolysate are hydrophobic amino acids, in particular alanine, leucine and isoleucine.

As an alternative to enzymatic hydrolysis, within the scope of the invention collagen hydrolysate may be produced by recombinant gene expression. By using natural collagen sequences, in particular from cattle or pigs, and expressing them in genetically modified cells (e.g. yeasts, bacteria or plant cells, in particular tobacco), it is possible to produce products that are substantially identical to the hydrolysis products of the corresponding raw materials containing collagen. In this case, it is possible to obtain a narrower or exactly predetermined molecular weight distribution.

According to a further embodiment of the invention, collagen hydrolysate is administered combined with at least one further active substance that counters shortening of telomeres in the cells of the body. In some circumstances, a combination of this kind enables even more pronounced effects to be achieved.

The at least one further active substance that counters shortening of telomeres is preferably selected from plant extracts of plants of the genera Astralagus (tragacanth), Cimicifuga (bugbane), Dendropanax and Kappaphycus.

According to a further embodiment of the invention, collagen hydrolysate is administered in a composition which does not contain any further active substances other than collagen hydrolysate. In particular, it may be provided for the administered composition to consist substantially of the entirely collagen hydrolysate.

Where the collagen hydrolysate is not used as the sole active substance, according to a further embodiment of the invention it may be combined with one or more further components (e.g. in a nutritional supplement or medicament) that have a positive physiological effect. Components of this kind are preferably selected from vitamin C, vitamins in the B, D, E and K series, conjugated linoleic acids, caffeine and derivatives thereof, guarana extract, green tea extract, epigallocatechin gallate, creatine, L-carnitine, L-citrulline, L-arginine, α-lipoic acid, N-acetylcysteine, NADH, D-ribose, magnesium aspartate, antioxidants such as anthocyanins, carotenoids, flavonoids, resveratrol, glutathione, superoxide dismutase and xanthans such as mangiferin, minerals such as iron, magnesium, calcium, zinc, selenium and phosphorus, and further proteins, hydrolysates or peptides such as soy, wheat or whey protein.

Further, the present invention relates to a method for delaying the ageing of cells in the bodies of humans or animals, comprising the administration of collagen hydrolysate to a human or an animal. Advantages and preferred embodiments of this method have already been described in conjunction with the collagen hydrolysate according to the invention.

The effectiveness of collagen hydrolysate against the shortening of telomeres is explained in more detail with reference to the examples below.

The in vitro tests on human fibroblasts that are described below, regarding the effects of collagen hydrolysate on telomere length and the activity of telomerase, were carried out by Life Length in Madrid, Spain.

### Example 1

1. Effect of Collagen Hydrolysate on Telomere Length

1.1 Cell Cultures

All the tests were performed on cell cultures of adult human fibroblasts. Inoculation was carried out in each case with 2 000 cells per cm² in high-glucose DMEM (Dulbecco's Modified Eagle's Medium) supplemented with 10% foetal calf serum, 100 units/ml of penicillin and 1 000 units/ml of streptomycin. The medium was replaced every 2 to 3 days, and the cells were passaged at a confluence of 70 to 80%, every 4 to 5 days.

In parallel with the cell cultures under standard conditions, cell cultures were also tested under oxidative conditions, in which the above-mentioned culture medium additionally contained 10 μM of H₂O₂.

1.2 Collagen Hydrolysate

The collagen hydrolysate (KH) used for the tests is sold by the applicant under the name VERSIOL®. It is produced by enzymatic hydrolysis from type A gelatine from pork rind, and has an average molecular weight of approximately 2 000 Da.

The effects of the collagen hydrolysate were tested over a broad range of concentrations. For this, in each case cell cultures in which 0.01 mg/ml, 0.1 mg/ml, 1 mg/ml or 10 mg/ml of collagen hydrolysate was added to the culture medium were grown (under both standard and oxidative conditions). Respective control batches had no collagen hydrolysate added.

1.3 Population Doubling

The cell cultures were grown for a period of 8 weeks, wherein the population doubling (PD) for each passage was determined weekly.

The cumulative PD is indicated in the tables below for standard conditions and oxidative conditions.

Cumulative PD for each passage under standard conditions

| KH [mg/ml] | 0 | 0.01 | 0.1 | 1 | 10 |
|---|---|---|---|---|---|
| Week 1 | 3.2 | 2.9 | 2.6 | 3.4 | 2.6 |
| Week 2 | 6.7 | 6.5 | 6.8 | 6.8 | 6.2 |
| Week 3 | 10.2 | 9.7 | 10.1 | 10.3 | 9.5 |
| Week 4 | 13.5 | 13.2 | 13.9 | 13.7 | 12.6 |
| Week 5 | 16.8 | 16.2 | 16.8 | 16.5 | 15.9 |
| Week 6 | 19.5 | 18.4 | 19.7 | 19.5 | 18.7 |
| Week 7 | 23.1 | 22.5 | 23.2 | 23.2 | 22.5 |
| Week 8 | 25.2 | 24.7 | 25.2 | 24.8 | 24.5 |

Cumulative PD for each passage under oxidative conditions

| KH [mg/ml] | 0 | 0.01 | 0.1 | 1 | 10 |
|---|---|---|---|---|---|
| Week 1 | 3.2 | 2.9 | 2.9 | 2.7 | 2.7 |
| Week 2 | 6.2 | 5.9 | 6.2 | 5.4 | 6.0 |
| Week 3 | 9.2 | 8.9 | 9.5 | 8.7 | 8.5 |
| Week 4 | 8.7 | 11.9 | 12.3 | 12.6 | 11.4 |
| Week 5 | 13.6 | 13.5 | 14.3 | 14.3 | 13.2 |
| Week 6 | 15.3 | 16.7 | 16.9 | 14.4 | 14.7 |
| Week 7 | 18.1 | 20.0 | 20.0 | 16.5 | 18.2 |
| Week 8 | 19.8 | 21.0 | 21.0 | 17.5 | 19.5 |

As expected, population doubling under oxidative conditions is consistently less than under standard conditions. By contrast, the presence of collagen hydrolysate has no significant effect on the PD, that is to say that cell growth per se is not affected by collagen hydrolysate.

1.4 Determination of Telomere Lengths and Shortening Rates

Telomere lengths are determined by fluorescence in-situ hybridisation (FISH). For this, a fluorescence-marked peptide nucleic acid probe hybridised with three repetitive telomere sequences is used. The intensity of fluorescence of a given telomere is proportional to its length, quantification being carried out with the aid of control cells of known telomere length.

In all the cell cultures tested, the telomere lengths were determined after 4 weeks and after 8 weeks, and in the control batch it was determined at the start of cultivation (week 0). The cells were fixed and underwent lysis using pepsin, wherein five cell lyses were performed for each point in time and each cell culture. The DNA was stained with 4′,6-diamidino-2-phenylindole (DAPI), and hybridisation of the telomeres was carried out using the probe.

Then the DAPI-stained cell nuclei were localised using a fluorescence microscope, and the fluorescence of the hybridised telomeres was quantified, wherein 15 different image sections were evaluated for each batch (cell lysis). The telomere lengths and distribution were calculated using appropriate software and underwent statistical analysis using Student's t-test.

1.5 Results

1.5.1 Telomere Lengths

The median value of the telomere length, the twentieth percentile (in each case in kilobase pairs, kbp) and the proportion of “short telomeres” (less than 3 kbp) were calculated for each cell culture after 4 weeks and 8 weeks from the data obtained by fluorescence microscopy. The results are indicated in the tables below. Also indicated in each case is the p value according to the t-test, in which the respective sample is compared with the corresponding sample of the control batch with no collagen hydrolysate. The difference from the control batch is statistically significant where the values are p<0.05; these values are shown in bold type.

Telomere lengths under standard conditions

| KH | | Median length | | 20th percentile | | Proportion <3 kbp | |
|---|---|---|---|---|---|---|---|
| mg/ml | Week | kbp | p value | kbp | p value | % | p value |
| 0 | 0 | 7.726 | — | 4.063 | — | 12.8 | — |
| 0 | 4 | 6.878 | — | 3.342 | — | 17.3 | — |
| 0.01 | 4 | 7.082 | 0.02 | 3.464 | 0.08 | 16.7 | 0.1 |
| 0.1 | 4 | 7.051 | 0.08 | 3.433 | 0.2 | 16.9 | 0.4 |
| 1 | 4 | 6.921 | 0.6 | 3.351 | 0.9 | 17.3 | 1.0 |
| 10 | 4 | 7.264 | 0.001 | 3.757 | 0.0004 | 14.6 | 0.0008 |
| 0 | 8 | 5.578 | — | 2.489 | — | 24.2 | — |
| 0.01 | 8 | 5.900 | 0.0003 | 2.736 | 0.006 | 22.0 | 0.005 |
| 0.1 | 8 | 5.884 | 0.003 | 2.862 | 0.002 | 21.0 | 0.002 |
| 1 | 8 | 5.830 | 0.002 | 2.875 | 0.0002 | 20.9 | 0.0003 |
| 10 | 8 | 5.846 | 0.1 | 2.905 | 0.009 | 20.6 | 0.008 |

Telomere lengths under oxidative conditions

| KH | | Median length | | 20th percentile | | Proportion <3 kbp | |
|---|---|---|---|---|---|---|---|
| mg/ml | Week | kbp | p value | kbp | p value | % | p value |
| 0 | 4 | 6.947 | — | 3.507 | — | 16.3 | — |
| 0.01 | 4 | 6.747 | 0.08 | 3.152 | 0.01 | 18.8 | 0.01 |
| 0.1 | 4 | 6.831 | 0.3 | 3.126 | 0.01 | 18.9 | 0.01 |
| 1 | 4 | 6.864 | 0.5 | 3.288 | 0.08 | 18.0 | 0.07 |
| 10 | 4 | 6.770 | 0.1 | 3.313 | 0.06 | 17.6 | 0.06 |
| 0 | 8 | 5.900 | — | 2.655 | — | 22.6 | — |
| 0.01 | 8 | 6.117 | 0.009 | 3.049 | 0.0001 | 19.5 | 0.0001 |
| 0.1 | 8 | 6.322 | 0.002 | 3.207 | 0.0003 | 18.1 | 0.0002 |
| 1 | 8 | 6.214 | 0.004 | 3.076 | 0.0006 | 19.3 | 0.0003 |
| 10 | 8 | 5.371 | 0.002 | 2.674 | 0.9 | 23.2 | 0.6 |

These data show a significant effect by collagen hydrolysate on the telomere lengths, at least after the fibroblasts have been cultivated for 8 weeks. This effect applies both under standard conditions and also in the presence of H₂O₂, and for all the concentrations tested.

1.5.2 Telomere Shortening Rates

For calculation of the telomere shortening rates, for each cell culture the telomere length after 4 weeks and after 8 weeks, minus the telomere length at the start of cultivation, was divided by the respective PD value (see section 1.3). Thus, the rate indicates the average shortening of the telomeres per population doubling.

The results are indicated in the tables below, and here too there is statistical significance in a comparison with the control batch where the p value is below 0.05.

Telomere shortening rates under standard conditions

| KH [mg/ml] | Week | Rate [bp/PD] | p value |
|---|---|---|---|
| 0 | 4 | 63.1 | — |
| 0.01 | 4 | 49.0 | 0.03 |
| 0.1 | 4 | 48.5 | 0.05 |
| 1 | 4 | 59.0 | 0.5 |
| 10 | 4 | 36.6 | 0.002 |
| 0 | 8 | 85.2 | — |
| 0.01 | 8 | 73.8 | 0.0007 |
| 0.1 | 8 | 73.1 | 0.003 |
| 1 | 8 | 76.6 | 0.005 |
| 10 | 8 | 76.8 | 1.0 |

Telomere shortening rates under oxidative conditions

| KH [mg/ml] | Week | Rate [bp/PD] | p value |
|---|---|---|---|
| 0 | 4 | 89.4 | — |
| 0.01 | 4 | 82.4 | 0.5 |
| 0.1 | 4 | 73.0 | 0.2 |
| 1 | 4 | 68.5 | 0.1 |
| 10 | 4 | 84.1 | 0.6 |
| 0 | 8 | 92.1 | — |
| 0.01 | 8 | 76.7 | 0.0001 |
| 0.1 | 8 | 66.8 | 0.0006 |
| 1 | 8 | 86.4 | 0.2 |
| 10 | 8 | 120.7 | 0.001 |

These data show a significant effect by collagen hydrolysate on the telomere shortening rates, at least after the fibroblasts have been cultivated for 8 weeks. This effect applies both under standard conditions and also in the presence of H₂O₂, and for all the concentrations tested.

### Example 2

2. Effect of Collagen Hydrolysate on Telomerase Activity

2.1 Cell Cultures

The test was carried out on primary cultures of adult human fibroblasts. The culture medium was high-glucose DMEM (Dulbecco's Modified Eagle's Medium) supplemented with 10% foetal calf serum, 100 units/ml of penicillin and 1 000 units/ml of streptomycin.

Telomerase activity was determined at the start of cultivation and after 24 hours.

2.2 Collagen Hydrolysate

The same collagen hydrolysate was used as that for determining telomere lengths (see section 1.2). A quantity of 0.01 mg/ml, 0.1 mg/ml, 1 mg/ml or 10 mg/ml of this was added to the culture medium. No collagen hydrolysate was added to the control batch.

2.3 Determining Telomerase Activity

Activity of the enzyme telomerase was determined by the TRAP method (telomeric repeat amplification protocol). For this, the cells were lysed and the proteins extracted. An oligonucleotide substrate was added, and this was extended by the extracted telomerase in a manner corresponding to natural telomeres.

The reaction products of telomerase were amplified using a quantitative real-time PCR. The measurement variable was the number of cycles after which fluorescence first exceeded a threshold value (Ct value). Calibration with the aid of different concentrations of HeLa cells made it possible to calculate the relative telomerase activity (RTA) of the sample.

2.4 Results

The relative telomerase activity in the fibroblasts at the start of cultivation and for the different KH concentrations after 24 hours were each determined three times. The table below indicates the average values and standard deviations (sd) as well as the p values according to the t-test for the comparison in each case with the control batch (with no KH). There is statistical significance if the p value is below 0.05.

Relative telomerase activity

| KH [mg/ml] | Hours | RTA average | sd | p value |
|---|---|---|---|---|
| 0 | 0 | 1.9 | 0.3 | — |
| 0 | 24 | 1.4 | 0.4 | — |
| 0.01 | 24 | 0.9 | 0.2 | 0.6 |
| 0.1 | 24 | 1.6 | 0.5 | 0.03 |
| 1 | 24 | 2.5 | 0.4 | 0.3 |
| 10 | 24 | 2.1 | 1.0 | 0.2 |

These data show that telomerase activity in the fibroblasts is increased by the addition of collagen hydrolysate in concentrations of 0.1, 1 or 10 mg/ml, although the increase is only significant in the case of 0.1 mg/ml.

1-20. (canceled)

21. A method for delaying the ageing of cells in the body of a human or an animal, comprising the administration of collagen hydrolysate to the human or the animal.

22. The method according to claim 21 for delaying the ageing of the skin, wherein the cells of the body are fibroblasts.

23. The method according to claim 21, wherein the method is a non-therapeutic method.

24. The method according to claim 21, wherein the method is a therapeutic method to prevent and/or treat age-related diseases.

25. The method according to claim 21, wherein the collagen hydrolysate counters the shortening of telomeres in the cells of the body, and/or reduces the telomere shortening rate.

26. The method according to claim 25, wherein the collagen hydrolysate activates the enzyme telomerase in the cells of the body.

27. The method according to claim 21, wherein the collagen hydrolysate is administered orally.

28. The method according to claim 27, wherein the collagen hydrolysate is administered in the form of a powder, a solution, a gel, a tablet or a capsule.

29. The method according to claim 21, wherein the collagen hydrolysate is administered in a daily dose of from approximately 0.5 to approximately 20 g.

30. The method according to claim 21, wherein the collagen hydrolysate has an average molecular weight of from 500 to 15 000 Da.

31. The method according to claim 21, wherein the collagen hydrolysate is produced by enzymatic hydrolysis of a starting material containing collagen.

32. The method according to claim 31, wherein the collagen-containing starting material is selected from the skin or bones of vertebrates.

33. The method according to claim 31, wherein the collagen hydrolysate is produced by the successive action of at least two endoproteases of different specificities.

34. The method according to claim 33, wherein the metalloproteases and/or serine proteases are selected from enzymes from the microorganisms Bacillus subtilis, Bacillus licheniformis, Bacillus amyloliquefaciens, Aspergillus oryzae and Aspergillus melleus.

35. The method according to claim 34, wherein at least 50% of the N-terminal amino acids of the collagen hydrolysate are hydrophobic amino acids.

36. The method according to claim 21, wherein the collagen hydrolysate is produced by recombinant gene expression.

37. The method according to claim 21, wherein the collagen hydrolysate is administered combined with at least one further active substance that counters shortening of telomeres in the cells of the body.

38. The method according to claim 37, wherein the at least one further active substance is selected from plant extracts of plants of the genera Astralagus (tragacanth), Cimicifuga (bugbane), Dendropanax and Kappaphycus.

39. The method according to claim 21, wherein the collagen hydrolysate is administered in a composition which does not contain any further active substances other than the collagen hydrolysate.

40. The method according to claim 39, wherein the composition administered consists substantially entirely of the collagen hydrolysate.
